(19) United States
(12) Patent Application Publication
(10) Pub. No.: US 2010/0204722 A1
(43) Pub. Date: Aug. 12, 2010

(54) APPARATUS FOR CUTTING OUT AND REMOVING TISSUE CYLINDERS FROM A TISSUE, AND USE THEREOF

(76) Inventor: John B. Gilsdorf, Atlanta, GA (US)

Correspondence Address: Peter G. Pappas;SUTHERLAND ASBILL & BRENNAN LLP, 999 Peachtree Street, NE, Atlanta, GA, 30309-3996, US

(21) Appl. No.: 12/621,295

(22) Filed: Nov. 18, 2009

(30) Foreign Application Priority Data
May 18, 2007 (DE) 102007023207.3
Oct. 17, 2007 (DE) 102007049796.4
May 19, 2008 (EP) PCT/EP08/04001

(51) Int. Cl. A61B 17/32

(52) U.S. Cl. 606/170

(57) ABSTRACT

The invention relates to an apparatus for cutting out and removing tissue cylinders from a tissue located within a cavity of the body or a joint and/or in or on a wall section thereof. The apparatus includes at least two, especially two cutting devices, each of which has a hollow cylindrical base, a distal opening at a distal end of the base, and a cutting element that surrounds the distal opening. The at least two cutting devices are disposed inside each other and can be rotated relative to one another. The apparatus further includes at least one driving device for rotating the respective base of the at least two cutting devices about the longitudinal axis of the base. The invention also relates to the use of the apparatus.

The invention relates to an apparatus for cutting out and removing tissue cylinders from a tissue located within a cavity of the body or a joint and/or in or on a wall region thereof, and use of same.

Such apparatuses are generally known. Accordingly, such apparatuses are used, simultaneously with a trocar and a trocar sleeve, for example in the medical field of gynecology, for removing tissue from a uterus or an entire uterus, or for treating myomas, etc. Such an apparatus is known from EP 0 522 125 B1, for example. These types of apparatuses are also used in the medical field for numerous applications and sometimes in other specialty areas, for example in surgical procedures in the abdominal cavity, the stomach, or for gall bladder resection or appendectomy. However, in some respects such apparatuses have proven to be relatively disadvantage in practice. All of these types of apparatuses have only one cutting device with a hollow cylindrical base body, a distal opening at a distal end of the base body, and a cutting element which surrounds the distal opening. The hollow cylindrical base body together with the cutting element is set in rotation in order to cut or punch out the tissue cylinder. However, as the result of such a rotation of the hollow cylindrical base body, the tissue from which the tissue cylinder is to be cut or punched out is at the same time deformed or sometimes even set in independent rotation. To allow a targeted intervention in the tissue, additional medical instruments, for example gripping instruments such as claw grasping forceps, are therefore necessary to correspondingly deform or fix the tissue in place. As a result, the handling of such apparatuses as a whole is complicated, and is more invasive and time-consuming for the patient.

The object of the invention, therefore, is to provide an apparatus for cutting out and removing tissue cylinders from a tissue, by means of which the above disadvantages may be prevented, and which thus has a particularly simple, compact, and stable design, and at the same time allows simple and rapid removal of a cut or punched tissue cylinder from the surgical area, ensures particularly simple, gentle, and above all time-saving handling, guarantees greatly enhanced and increased patient safety, and provides advantageous use of this apparatus.

With respect to the apparatus, this object is achieved in a surprisingly simple manner by means of the features of Claim 1.

Accordingly, as a result of the design of the apparatus according to the invention for cutting out and removing tissue cylinders from a tissue located within a cavity of the body or a joint and/or in or on a wall region thereof, comprising at least two, in particular two, cutting devices, each of which has a hollow cylindrical base body, a distal opening at a distal end of the base body, and a cutting element which surrounds the distal opening, the at least two cutting devices being situated one inside the other and designed to be rotatable relative to one another, and at least one drive device for rotating the respective base body of the at least two cutting devices about the longitudinal axis of the base body, it is possible to achieve a simple, compact, and stable design of the overall apparatus. Furthermore, the apparatus according to the invention, which may also be referred to as a morcellator, has the significant advantage that during cutting or punching out of a tissue cylinder the apparatus does not require an additional instrument, in particular a gripping instrument such as claw grasping forceps or the like, for deforming, or holding the tissue during the surgical procedure. Rather, as a result of the design of the apparatus according to the invention with at least two, in particular two, such cutting elements, deformation or independent rotation of the tissue from which a tissue cylinder to be morcellated is to be cut or punched out is prevented. Not least of all, such a design results in particularly simple, very gentle, and above all time-saving handling of the apparatus as a whole. Lastly, greatly enhanced and increased patient safety may be achieved using the apparatus according to the invention.

Further advantageous particulars of the apparatus according to the invention are described in Claims 2 through 41.

According to Claim 2 it is within the scope of the invention that the inner cutting device is coaxially accommodated by the adjacent outer cutting device of the at least two cutting devices 2.

According to Claim 3, the inner cutting device is advantageously accommodated by the adjacent outer cutting device of the at least two cutting devices with little play.

Alternatively, according to Claim 4 the inner cutting device is accommodated by the adjacent outer cutting device of the at least two cutting devices with greater play, wherein at least one centering and/or guide element is situated between the base bodies of the inner cutting device and the adjacent outer cutting device. Coaxial alignment of the cutting devices mutually adjoining the base body is thus achieved by use of this centering and/or guide element.

According to Claim 5, it is further within the scope of the invention that the at least two cutting devices may be driven by the drive device in counterrotation and/or in corotation. Of primary importance in this regard is the design according to the invention of a counterrotating drive of the at least two cutting devices relative to one another by use of the drive device. Namely, use of a drive in counterrotation also makes it possible for the tissue from which the tissue cylinder is to be cut or punched out to not be subjected to deformation or even independent rotation when it is acted on by the cutting device. Rotation of the at least two, in particular two, cutting devices relative to one another causes the threes and torques acting on the tissue during cutting or punching out of the tissue cylinder to essentially cancel one another out.

For this purpose, according to Claim 6 gearing which is accommodated in a housing is advantageously provided between the at least two cutting devices and the drive device.

In a further embodiment of the invention, according to the measures of Claim 7 the gearing includes an output gear wheel associated with the drive device, and two input gear wheels, in operative engagement with the one output gear wheel, which are each associated with one of the at least two cutting devices.

According to Claim 8, the output gear wheel and the two input gear wheels of the gearing are preferably designed as bevel gears.

The features of Claim 9 serve to simplify the design of the apparatus according to the invention. Accordingly, the two input gear wheels which are each associated with one of the at least two cutting devices are in simultaneous operative engagement with the output gear wheel associated with the drive device. The rotational axes of the two input gear wheels coincide. In contrast, the rotational axes of the two input gear wheels are each offset by ±90° relative to the rotational axis of the output gear wheel. By use of such a design the at least two cutting devices may be easily driven in counterrotation to one another. The drive may also be provided using only a single drive device.

According to Claim 10, it is further provided according to the invention that the output gear wheel is advantageously detachably mounted on a support element which is rotatably supported on the housing of the gearing. As a result of such a two-part design of the output gear wheel, the support element, which generally is acted on with considerable forces and/or torques by the drive device for transmitting the rotational motion, may be made of a more wear-resistant material. Such a two-part design of the output gear wheel allows simple, rapid, and therefore economical replacement when the output gear wheel wears out.

According to Claim 11, to simplify installation and/or repair the output gear wheel and the support element are advantageously detachably joined together by a locking or snap connection or a threaded connection. Additional securing may be provided by pins, etc.

According to Claim 12 the output gear wheel and, if present, the support element together with the drive device, is connected in a rotationally fixed manner via a peg-shaped transmission element to a profile having a rectangular, square, hexagonal, or otherwise polygonal, elliptical, trapezoidal, parallelogram-like, or semicircular shape.

According to Claim 13, it is further within the scope of the invention that the two input gear wheels are each accommodated by drive bushes which are respectively associated with one of the at least two cutting devices. By use of such a two-part design it is likewise possible to select a different material for the input gear wheels and the drive bushes, depending on the particular demands. In addition, replacement for repair of the drive wheels is therefore simple, rapid, and economical.

According to Claim 14, the two input gear wheels are each attached to the drive bushes via a threaded connection or a locking or snap connection. Although not specifically illustrated, separate securing, for example using pins, etc may also be provided.

By use of the measures according to Claim 15, the drive bushes advantageously project through the housing of the gearing, thus providing a constructive connection between the input gear wheels and the respectively associated at least two cutting devices.

According to Claim 16, it is further within the scope of the invention that the drive device may be controlled and/or regulated using a control and/or regulation device which in particular is electronic, in such a way that the at least two cutting devices may each be driven by the drive device at intervals in the opposite drive direction. This ensures that two mutually adjoining cutting devices may be driven in any given manner in a right-left rotation and/or in reverse, and likewise in any given manner in a left-right rotation.

In this regard, according to Claim 17 it is advantageous when the time intervals for driving the at least two cutting devices in the opposite drive direction may be set and/or preset by a control and/or regulation device which in particular is electronic.

According to the features of Claim 18, the at least two cutting devices are each provided with coupling elements at the proximal end and/or at the region of the base body facing the proximal end, the coupling elements being nondetachably connected to the respective base body.

The design measures of Claim 19 are of particular interest. Accordingly, the at least two cutting devices and the drive device together with the gearing may each be connected in a rotationally fixed manner via a coupling device. The coupling device has a constructive design which is particularly easy for the user to handle.

For this purpose, according to Claim 20 the coupling device is designed as a plug-in, locking, or snap device. Such a plug-in, locking, or snap device is particularly advantageous because during the surgical procedure the at least two cutting devices and the drive device may be easily and rapidly connected to the gearing or subsequently disconnected, and if need be, even with only hand of the user.

In this regard, according to Claim 21 it is particularly advantageous that the coupling device has a rocker arm which is mounted in each case on the drive bushes and the housing of the gearing so as to be pivotable about a rotational axis, at one end is acted on by a spring, and at an end opposite from the one end includes an approximately radially situated pin, bolt, or projection for engaging in a correspondingly designed borehole or recess in the coupling element of the at least two cutting devices or the drive device.

According to Claim 22, it is further within the scope of the invention that the coupling element of the inner/innermost of the at least two cutting devices is designed as a sealing device for closing and exposing a proximal opening at the proximal end of the base body.

According to the features of Claim 23, the sealing device advantageously has a two-part, essentially cup-shaped housing with a housing element mounted on the base body, and a lid- or cap-shaped closure element which may be fastened to the housing element.

The design measures of Claim 24 very advantageously provide simple, rapid, reliable, and in particular economical manufacture and installation. Accordingly, the housing element and the closure element of the housing are detachably or nondetachably connected to one another via a threaded connection or a locking or snap connection. The constructive design of providing a detachable or nondetachable connection between the housing element and the closure element of the housing depends largely on the intended use and purpose of the apparatus according to the invention, i.e., whether the apparatus is to be provided for single or multiple use. This in turn has a great effect on the design of the apparatus as a whole or the selection of materials of which the individual components of the apparatus according to the invention are composed, and vice versa.

If, for example, the apparatus is intended only for a single use, i.e., it is completely disposed of after use, according to Claim 25 the housing element and the closure element of the housing are nondetachably joined together, preferably by providing an adhesive, in particular a silicone adhesive, in between, or by ultrasonic welding.

On the other hand, for multiple use the housing element and the closure element of the housing, which in that case are most likely made of metal, are screwed together via a thread, for example.

As an alternative to the threaded connection, a locking or snap connection may be provided in which according to Claim 26 the housing element and the closure element of the housing are connected to one another via at least uric locking projection or similar catch lock on the housing element and at least one additional locking projection or similar catch lock on the closure element, which cooperate and may be brought into mutual engagement. A kinematic reversal of same by providing the at least one locking projection or similar catch lock on the closure element, and the at least one catch recess or the like on the housing element, is also possible. Instead of the at least one additional locking projection, at least one catch recess or the like may be provided which cooperates with the at least one locking projection.

For a single use, in this regard the apparatus according to the invention is advantageously designed corresponding to the features of Claim 27. Accordingly, the at least one locking projection or similar catch lock is provided on the housing element or on the closure element, and the at least one additional locking projection or similar catch lock is provided on the closure element or on the housing element in such a way that the housing element and the closure element of the housing are either on the one hand nondetachably connected to one another after engaging the at least one locking projection or similar catch lock and the at least one additional locking projection or similar catch lock, or on the other hand are not connectable to one another after disengaging the at least one locking projection or similar catch lock and the at least one additional locking projection or similar catch lock. In the latter design it would be possible, for example, to provide one of the two locking projections or similar catch locks with a predetermined breaking point so that this locking projection or similar catch lock breaks away from the housing element or closure element when acted on by a given tensile force. Instead of the at least one additional locking projection, at least one catch recess or the like may be provided which cooperates with the at least one locking projection.

Also of great importance are the design measures of Claim 28, according to which the sealing device includes two sealing elements which are adjacently situated in the housing on the longitudinal axis of the base body of the cutting device, and which cooperate with one another.

According to Claim 29, it is provided according to the invention that one of the two sealing elements of the sealing device essentially has the shape of a cone, conical frustum, cup, hat, or the like, and includes a slotted opening situated in a plane which is offset with respect to a plane defined by the base area of the sealing element in the direction of the distal end of the base body of the cutting device, and for fixing in the housing has a radially outwardly extending peripheral section in the plane defined by the base area of the sealing element. On account of its shape and positioning in the housing, one of the two sealing elements is forced into and held in its closed position in the opposite direction of the action of a pressure artificially developed within a cavity of the body or a joint, for example by CO₂ insufflation. The sealing element is externally pressed with a complete seal against a further instrument or shaft thereof, for example a gripping or other cutting instrument, in particular claw grasping forceps or a myoma drill. This ensures that the positive pressure necessary for the operation, i.e., cutting or punching out, a tissue cylinder from a tissue and for subsequent removal of the cut or punched tissue cylinder from the surgical area is maintained at all times. Release of gas such as CO₂ or the like from the cavity of the body or a joint in the surgical area is therefore prevented, thus counteracting hindrance of the peeling process or traumatizing the surrounding tissue during the peeling.

It is in particular within the scope of the invention that according to Claim 30, one of the two sealing elements has at least two sealing lips which are in mutual operative engagement for closing and exposing the slotted opening.

In addition, according to Claim 31 the other of the two sealing elements of the sealing device has a disk-shaped design, and has a central, approximately circular opening through which a further instrument, in particular a gripping or other cutting instrument, preferably claw grasping forceps or a myoma drill, may be passed.

According to Claim 32 the central, approximately circular opening in the other of the two sealing elements is preferably provided with an inner diameter which is less than or equal to an outer diameter of the further instrument.

It is further within the scope of the invention that in a constructive design according to Claim 33, the other of the two sealing elements in the housing is situated on the longitudinal axis of the base body of the cutting device, between the proximal end of the base body and the one of the two scaling elements, in particular adjacent to the plane defined by the base area of the one of the two sealing elements. This achieves mutual cooperation of the two sealing elements, thus enabling further improvement of the seal.

To improve the sealing effect, according to Claim 34 the at least one, in particular the two, sealing element(s) of the sealing device is/are made of flexible plastic, in particular silicone.

The features of Claim 35 are of particular interest. Accordingly, the cutting elements of the at least two cutting devices surrounding the distal opening essentially have a design which is beveled or conical, or which extends obliquely outward facing away from the distal opening and/or obliquely inward facing the distal opening.

In this regard, according to Claim 36 the cutting elements of the at least two cutting devices advantageously have a very sharp circumferential, i.e., continuous, or discontinuous smooth, serrated, or dentate face which makes a precise cut in the tissue when the base body undergoes a rotational motion, without causing excessive trauma to the remaining tissue. In this manner the tissue may be peeled without deforming the surrounding tissue during the procedure.

According to Claim 37, the cutting elements of the at least two cutting devices at the foremost edge of the distal opening are preferably provided with a serrated face. This embodiment of the invention results in multiple circular are sections distributed over the circumference with axially receding wave troughs and axially projecting wave crests. For example, for four wave projections and four wave troughs therebetween receding slightly axially in the circumferential surface, circular are sections approximately in the range of 40 to 45° would result. The serrated face may be present circumferentially or continuously, i.e., present over the entire circumference of the distal opening. The sharpening zone may advantageously have a particularly sharp design in the transition region between the wave trough and the wave projection, so that initially the tissue is axially fixed, and only then is the morcellation of tissue cylinders or tissue sleeves carried out due to the rotational motion of the base body. However, the serrated face may optionally be provided at the distal opening in a discontinuous manner with a scale-like design around the surface periphery.

The same applies to the alternative design of the invention according to Claim 38. Accordingly, the cutting elements of the at least two cutting devices with the dentate face have teeth with axially receding tooth bases and axially projecting tooth tips which are distributed over the circumference. Depending on the intended use, a circumferential, i.e., continuous, or discontinuous dentate face is possible.

For punching out or peeling tissue, according to Claim 39 the rotational motion may be applied to the base body via a motor which may be driven electrically, by battery, pneumatically, or hydraulically.

In a particularly advantageous manner, according to Claim 40 the housing of the gearing and/or the centering and/or guide element and/or the output gear wheel and/or the two input gear wheels and/or the support element and/or the drive bushes and/or the coupling elements and/or the sealing device and/or housing element and/or the closure element of the housing and/or the clamping disk may be made of plastic, in particular polyoxymethylene, polyester, polyphenylene sulfone, ABS, acrylic, polycarbonate, tetrafluoroethylene or impax, duroplastic elastomers with or without fiberglass reinforcement, or a combination thereof. The apparatus according to the invention is particularly lightweight, and may be manufactured very economically.

Alternatively, according to Claim 41 it is also within the scope of the invention for the base bodies of the at least two cutting devices and/or the base body of the at least two cutting devices [sic] and/or the housing of the gearing and/or the centering and/or guide element and/or the output gear wheel and/or one or both of the two input gear wheels and/or the support element and/or the drive bushes and/or the coupling elements and/or the sealing device and/or the coupling device and/or housing element and/or the closure element of the housing and/or the clamping disk to be made of metal, in particular (rust-free) steel, stainless steel, aluminum, brass, zinc, red brass alloys, or an alloy of the above.

Thus, the apparatus as a whole may be thermally and/or chemically sterilized and easily reused multiple times.

The design measures of Claim 42 are also of particular interest. Accordingly, the individual components of the gearing situated between the at least two cutting devices and the drive device are designed and may be assembled in a modular fashion. The gearing and therefore the entire apparatus may thus be put together in a modular fashion by the operator or service personnel. On the one hand, the manufacturer may realize additional cost savings for installation. On the other hand, the apparatus according to the invention may thus be easily, quickly, and reliably disassembled, for example for disinfection or the like, and then reassembled for reuse.

In this regard the features of Claim 43 are of great advantage, according to which the housing of the gearing or the housing element and the housing cover and/or the output gear wheel and/or one or both of the two input gear wheels and/or the support element are designed and may be assembled in a modular fashion.

lastly, with regard to use this object is further achieved by the features of Claim 44.

Accordingly, an apparatus for cutting out and removing tissue cylinders from a tissue located within a cavity of the body or a joint and/or in or on a wall region thereof, for example an organic body at least partially accommodated and/or surrounded by an organic cavity in a human body, preferably embryos, myomas, tumors, ulcers, and carcinomas. The apparatus according to the invention is particularly suited for laparascopic, thoracoscopic, or arthroscopic minimally invasive surgical procedures and the like. Although not specifically described, it is also possible with the aid of the apparatus according to the invention to cut out and remove inorganic bodies in the form of such body cylinders, such as gallstones and bladder stones or the like, present within a cavity of the body or a joint and/or in or on a wall region thereof.

Further features, advantages, and particulars of the invention result from the following description of one preferred embodiment of the invention, and with reference to the drawings, which show the following:

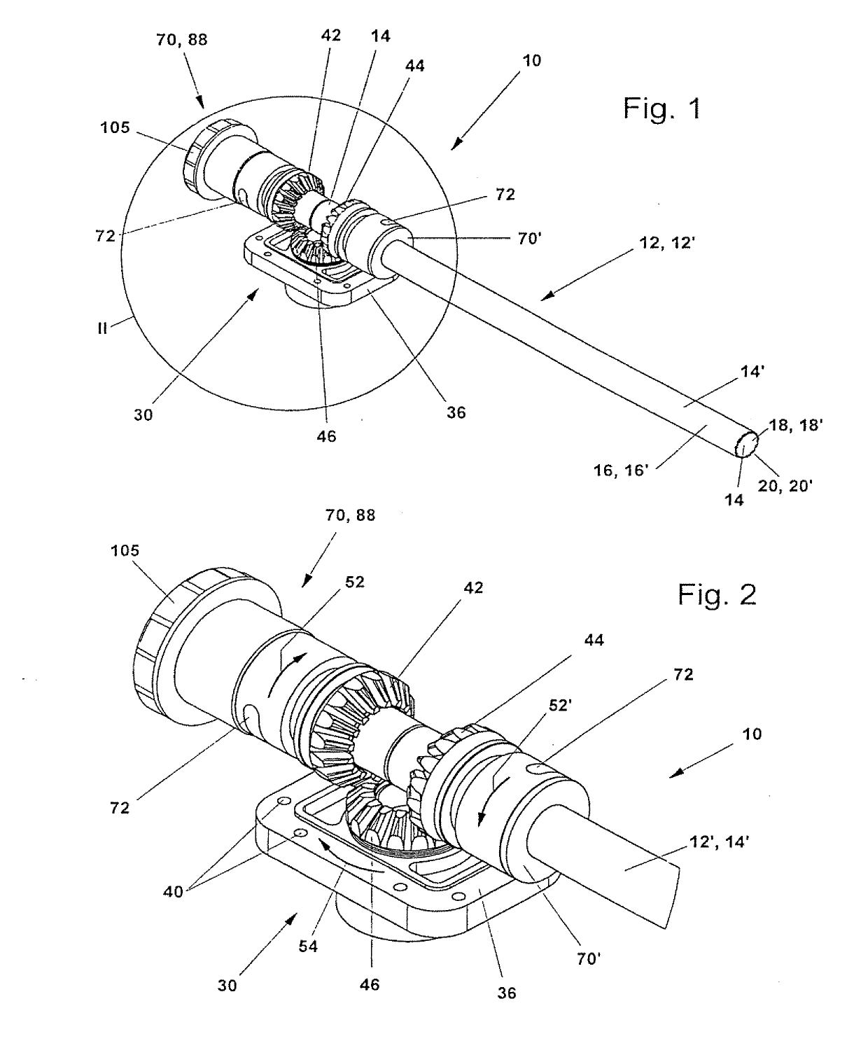

FIG. 1 shows a perspective front view of one embodiment of an apparatus according to the invention for cutting out and removing tissue cylinders from a tissue, without a housing;

FIG. 2 shows a partially truncated perspective view of the embodiment of the apparatus designed according to the invention corresponding to section II in FIG. 1, in an enlarged illustration;

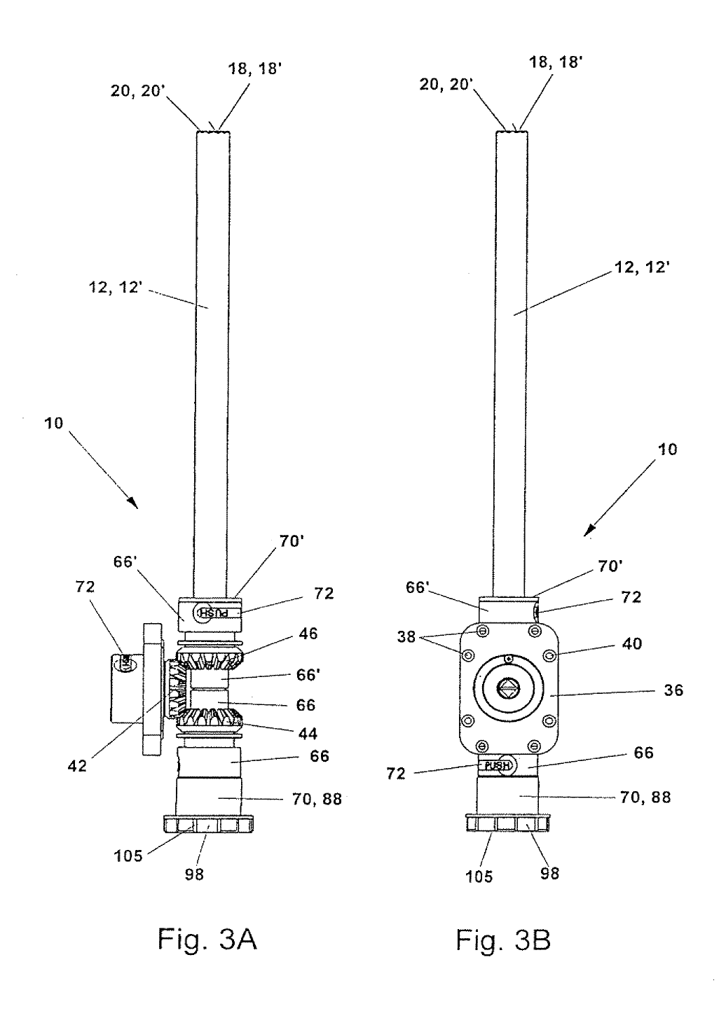

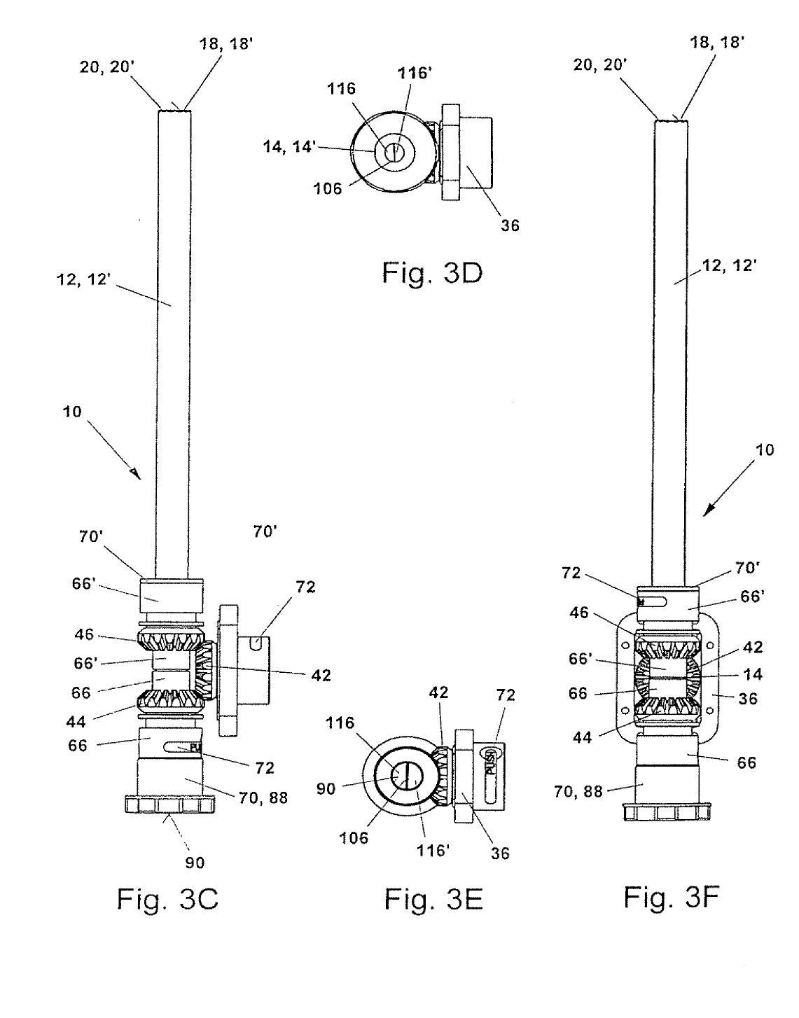

FIGS. 3A through 3F show a side view, a bottom view, a further side view, a front view, a rear view, and a top view of the embodiment of the apparatus designed according to the invention corresponding to FIGS. 1 and 2;

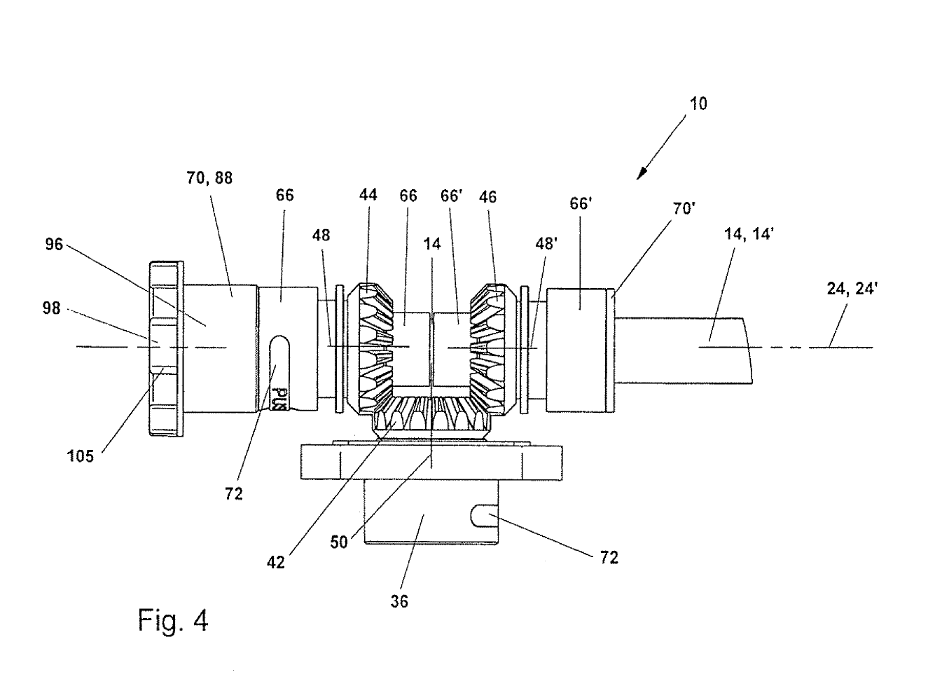

FIG. 4 shows a partially truncated perspective view of the embodiment of the apparatus designed according to the invention corresponding to FIG. 3C, in an enlarged illustration;

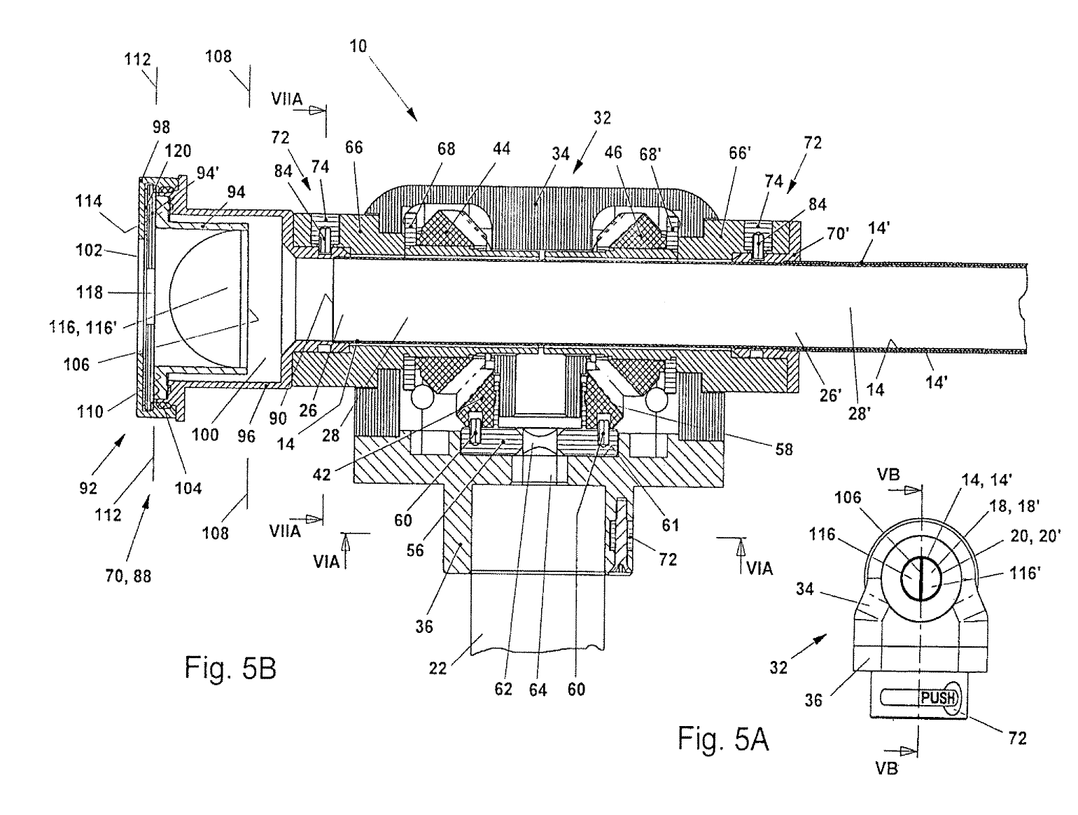

FIGS. 5A and 5B show a front view and a cross-sectional view of the embodiment of the apparatus designed according to the invention, along line VB-VB in FIG. 5A, with the housing, in an enlarged illustration;

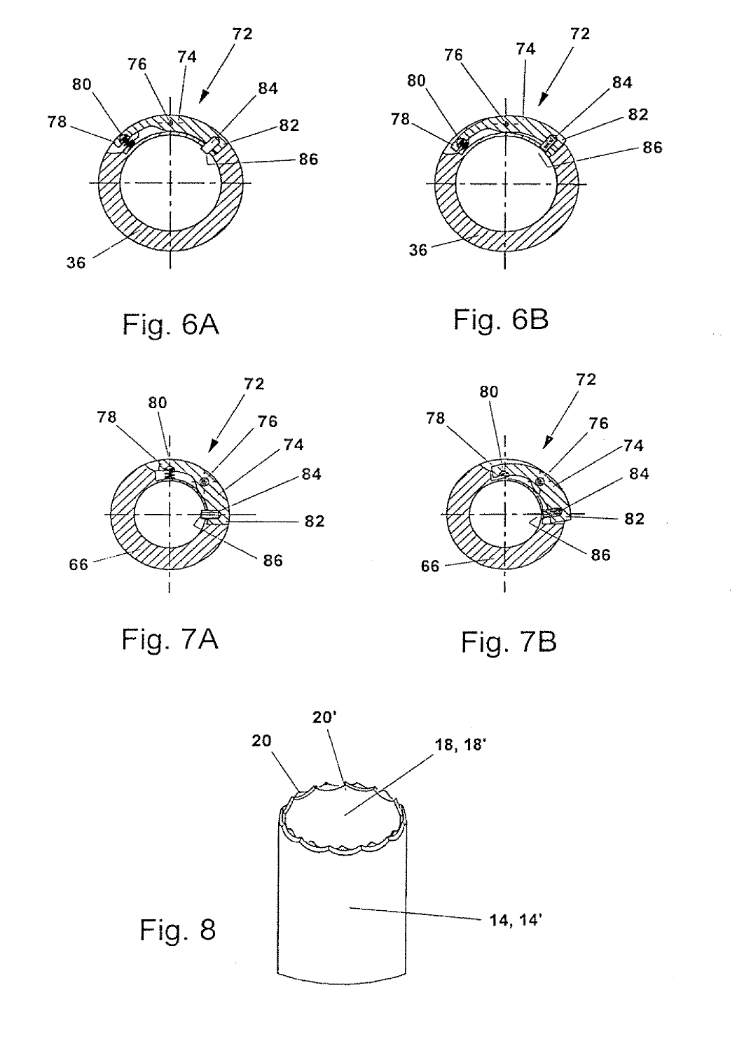

FIGS. 6A and 613 show schematic cross-sectional views of the embodiment of the apparatus designed according to the invention along line VIA-VIA in FIG. 5B, in the closed position and in the open position;

FIGS. 7A and 7B show schematic cross-sectional views of the embodiment of the apparatus designed according to the invention along line VIIA-VIIA in FIG. 5B in the closed position and in the open position; and

FIG. 8 shows a truncated, schematic perspective view of cutting elements having the design of the apparatus according to the invention according to FIGS. 1 through 7B, in an enlarged illustration.

The apparatus according to the invention 10 is provided for cutting out and removing tissue cylinders from a tissue located within a cavity of the body or a joint and/or in or on a wall region thereof. In the following description of one exemplary embodiment of the apparatus 10 according to the invention, corresponding identical parts are provided with the same reference numerals.

The apparatus 10 according to the invention is suited in the broadest sense for surgically operating on, in particular removing, an organic tissue, preferably myomas, tumors, ulcers, carcinomas, etc., or an inorganic body such as gallstones, bladder stones, or such agglomerations, which are at least partially accommodated and/or surrounded by a cavity of the body or a joint or an organic cavity in a human or animal body, and/or in or on a wall region thereof.

FIGS. 1 through 7B illustrate a preferred embodiment of such an apparatus 10 according to the invention.

The apparatus 10 comprises at least two cutting devices 12, 12′, each having a hollow cylindrical base body 14, 14′. A distal opening 18, 18′ is present at the distal end 16, 16′ of each base body 14, 14′. Each distal opening 18, 18′ is surrounded by a respective cutting element 20, 20′.

In the apparatus shown in FIGS. 1 through 7B, two such cutting devices 12, 12′ are provided, which corresponds to a particularly advantageous implementation of the invention. Although not specifically illustrated, in principle three or even more cutting devices 12, 12′ may also be provided.

The two cutting devices 12 and 12′ are provided one inside the other. In this regard, the inner cutting element 20 is accommodated by the outer cutting element 20′. In addition, the two cutting elements 20, 20′ are designed to be rotatable relative to one another.

The inner cutting device 12 is thus coaxially accommodated by the adjacent outer cutting device 12′ of the two cutting devices 12, 12′. There is preferably only a small amount of play between the inner cutting device 12 and the adjacent outer cutting device 12′. Therefore, the outer diameter of the base body 14 of the cutting device 12 is slightly smaller than the inner diameter of the base body 14′ of the cutting device 12′, and vice versa. The play between the inner cutting device 12 and the adjacent outer cutting device 12′ should be selected in such a way that the cutting devices 12, 12′ are able to easily rotate relative to one another while at the same time allowing precise guiding toward and relative to one another. In addition, the aim is to largely or substantially prevent passage of blood, bodily fluid, gas, etc. between the outer diameter of the base body 14 of the cutting device 12 and the inner diameter of the base body 14′ of the cutting device 12′.

Alternatively, although not specifically illustrated, for this purpose it would be possible for the inner cutting device 12 to be accommodated by the adjacent outer cutting device 12′ with even greater play. However, in such an embodiment one or more centering and/or guide elements (not illustrated) are provided between the base bodies 14, 14′ of the inner cutting device 12 and of the adjacent outer cutting device 12′. This/these centering and/or guide element(s) in particular is/are intended to prevent tilting and/or binding during the relative rotation of the inner cutting device 12 with respect to the adjacent outer cutting device 12′, and to prevent passage of blood, bodily fluid, gas, etc.

For rotation of the respective base body 14, 14′ of the two cutting devices 12, 12′ about its respective longitudinal axis 24, 24′, the apparatus 10 is provided with at least one drive device 22. In the present exemplary embodiment of the apparatus 10 only one such drive device 22 is provided (schematically illustrated). The drive device 22 may be situated at a proximal end 26 of one of the base bodies 14, 14′. Alternatively, or as shown in the present exemplary embodiment, the drive device 22 is situated at a region 28 of the base body 14 which faces and/or is adjacent to only the proximal end 26. The drive device 22 is designed as a motor which may be driven electrically, by battery, pneumatically, or hydraulically, for example.

In a very advantageous manner the two cutting devices 12, 12″ may be driven in counterrotation to one another by the drive device 22. Thus, in a particularly simple and therefore economical manner the tissue (not shown) from which the corresponding tissue cylinder is to be cut and/or punched Out is not deformed or set in independent rotation within a cavity of the body or a joint and/or in or on a wall region thereof during the surgical procedure using the apparatus 10 according to the invention. Due to the counterrotations of the two cutting devices 12, 12′, in any event the forces and/or any torques acting on the tissue as a result of the corresponding cutting elements 20, 20′ cancel one another out. As a result, during the surgical procedure using the apparatus 10 according to the invention the tissue is undeformed or remains in a given position without independent motion.

Although not specifically illustrated, the drive device 22 may be controlled and/or regulated by a control and/or regulation device (not illustrated) which in particular is electronic, so that the two cutting devices 12, 12′ may each be driven by the drive device 22 at intervals in the opposite drive direction. The change in the drive direction, which alternates in an oscillating or similar manner, for example, of the two cutting devices 12, 12′ is independent of the relative rotational motion of the cutting devices. In other words, the two cutting devices 12, 12′ may be driven in the opposite drive direction without eliminating the relative drive of the cutting devices in counterrotation and/or in corotation. This ensures that two mutually adjoining cutting devices 12, 12′ may be driven in any given manner in a right-left rotation and/or in reverse, and likewise in any given manner in a left-right rotation.

It is particularly advantageous when the time intervals for driving the two cutting devices 12, 12′ in the opposite drive direction may be set and/or preset by a control and/or regulation device which in particular is electronic. In this regard the time intervals may be selected to be the same or different, precisely adapted to the particular individual surgical area in question, stochastically formed, etc.

In order to drive the two cutting devices 12, 12′ in counterrotation and/or in corotation by the drive device 22, gearing 30 which is accommodated in a housing 32 is provided between the two cutting devices 12, 12′ and the drive device 22. The housing 32 is composed of a housing element 34, shown only in FIGS. 5A and 5B, and a housing cover 36, as shown in all of FIGS. 1 through 7B. The housing element 34 and the housing cover 36 may be secured to one another by means of screws 38 which pass through corresponding boreholes or openings in the housing element 34 (not illustrated) and tightened in corresponding boreholes 40 or openings in the housing cover 36 in a customary manner.

For driving in counterrotation, the gearing 30 includes an output gear wheel 42 which is associated with the drive device 22. The gearing 30 also has two input gear wheels 44, 46 which are in operative engagement with the output gear wheel 42 and are each associated with one of the two cutting devices 12, 12′.

In the embodiment of the apparatus 10 according to the invention illustrated in FIGS. 1 through 7B, the output gear wheel 42 and the two input gear wheels 44, 46 of the gearing 30 are designed as bevel gears.

In the embodiment of the apparatus 10 shown in FIGS. 1 through 7B, the two input gear wheels 44 and 46, which are each associated with one of the two cutting devices 12 or 12′, are in simultaneous operative engagement with the output gear wheel 42 which is associated with the drive device 44 [sic; 22]. In the present embodiment the rotational axes 48, 48′ of the two input gear wheels 44, 46 coincide. The rotational axes 48, 48′ of the two input gear wheels 44, 46 are also identical to the longitudinal axes 24, 24′ of the base bodies 14, 14′. In contrast, the rotational axes 48, 48′ of the two input gear wheels 44, 46 are offset relative to the rotational axis 50 of the output gear wheel 42, one by +90° and one by −90°. Such a constructive design achieves simultaneous, uniform, but also opposite rotation of the two input gear wheels 44, 46 corresponding to arrows 52, 52′ via rotation of the output gear wheel 42 corresponding to arrow 54. On the other hand, when the output gear wheel 42 rotates in the opposite direction from arrow 54, the rotational direction of the two input gear wheels 44, 46 also reverses corresponding to arrows 52, 52′ (not illustrated).

As shown in particular in FIGS. 2 and 5B, the output gear wheel 42 is also detachably fastened to an approximately disk-shaped support element 56. For this purpose the output gear wheel 44 and the support element 56 are detachably connected to one another via a locking or snap connection 58 in addition, pins 60, bolts, or the like are provided to prevent relative motion between the output gear wheel 42 and the support element 56.

The disk-shaped support element 56 is rotatably supported on the housing cover 36 of the housing 32 of the gearing 30. For example, the support element 56 is inserted into a circular opening 61. The support element 56 is further provided with a centrally situated opening 62 in which a peg-shaped transmission element 64 engages in a rotationally fixed manner. The centrally situated opening 62 and the transmission element 64 have corresponding designs. The transmission element 64 may be provided with a profile having, for example, a rectangular, square, hexagonal, or otherwise polygonal, elliptical, trapezoidal, parallelogram-like, or semicircular cross section.

The two-part constructive design of the output gear wheel 42 and the support element 56 allows, on the one hand, use of various materials having different characteristics. For example, in the present exemplary embodiment of the apparatus 10 the output gear wheel 42 is made of polyoxymethylene (POM), whereas the support element 56 is made of polyphenylene sulfone (PPSU). It is known that when high forces and/or torques occur which are transmitted from the drive device 22 via the transmission element 64 and act on the support element 56, polyphenylene sulfone is much more resistant and less prone to wear than many other plastics. On the other hand, such a constructive design allows simple, rapid, and therefore economical replacement when the output gear wheel 42 is worn out, or for other repairs.

The same applies for the two input gear wheels 44, 46, which in the present embodiment of the apparatus 10 shown in FIGS. 1 through 7B likewise have a two-part design. Accordingly, the two input gear wheels 44, 46 are accommodated by drive bushes 66, 66′, respectively, which are each associated with one of the two cutting devices 12, 12′.

According to FIG. 5B, for the illustrated apparatus 10 the two input gear wheels 44, 46 are attached to drive bushes 66, 66′, respectively, via a threaded connection. For securing, (flat or thrust) washers 68, 68′ are provided, against which the input gear wheels 44, 46 may be tightened. It is also possible to secure the input gear wheels 44, 46 using pins or bolts (not shown), or by providing an adhesive, preferably a silicone adhesive, in between, or by ultrasonic welding, although such additional securing may make rapid assembly or disassembly for repairs more difficult. As clearly shown in FIG. 5B, the drive bushes 66, 66′ themselves extend through the housing 32 of the gearing 30.

As likewise shown in FIG. 5B, the at least two cutting devices 12, 12′ are provided with coupling elements 70, 70′ which are nondetachably connected to the respective base body 14, 14′ of the cutting devices 12, 12′. The coupling elements 70, 70′ may be situated at the proximal end 26, 26′. Alternatively or additionally, the coupling elements 70, 70′ in the illustrated exemplary embodiment are situated in a region 28, 28′ of the base body 14, 14′ which faces the proximal end 26, 26′.

In addition, the two cutting devices 12, 12′ and the drive device 22 are each connected in a rotationally fixed manner to the gearing 30 via a coupling device 72. The coupling device 72 is designed as plug-in, locking, or snap connection.

For this purpose the coupling device 72 has a rocker arm 74. The rocker arm 74 is mounted in each case on the drive bushes 66, 66′ and the housing 32 of the gearing 30 so as to be pivotable about a rotational axis 76. At one end 78 the rocker arm 74 is acted on by a spring 80. At an end 82 oppositely situated from end 78 a pin 84, bolt, or projection is provided which cooperates with a correspondingly designed borehole 86 or opening in the associated coupling element 70, 70′ of the two cutting devices 12, 12′ or the drive device 22 (not illustrated). The pin 84, bolt, or projection and the associated borehole 86 or opening each extend approximately radially with respect to the longitudinal axes 24, 24′ of the base bodies 14, 14′ of the apparatus 10.

The spring 80 is situated in such a way that the rocker arm 74 is continuously held in the closed position of the coupling device 72. In the closed position illustrated in FIGS. 6A and 7A, the radially extending pin 84, bolt, or projection is engaged with the borehole 86 or opening. The respective cutting device 12, 12′ or the drive device 22 are thus connected to the gearing 30 in a rotationally fixed manner.

In contrast, in the open position of the coupling device 72, which is achieved by pressing down the rocker arm 64 against the force effect of the spring 80, the rocker arm 74 together with the pin 84, bolt, or projection is swiveled radially outward. As a result, the pin 84, bolt, or projection is disengaged from the borehole 86 or opening in the open position shown in FIGS. 6B and 7B. In this manner the cutting devices 12, 12′ or the drive device 22 may be easily separated from the gearing 30.

In the design of the apparatus 10 according to the invention shown in FIGS. 1 through 7B, the coupling element 70 associated with the inner cutting device 12 of the two cutting devices 12, 12′ is designed as a sealing device 88.

The sealing device 88 is used for closing and exposing a proximal opening 90 located at the proximal end 26 of the base body 14. The sealing device 88 includes a housing 92 and at least one sealing element 94, 94′ which may be accommodated in the housing 92. As shown in FIGS. 1 through 7B, in the illustrated embodiment of the apparatus 10 according to the invention a total of two sealing elements 94, 94′ are provided. Although not specifically illustrated, it is also possible to provide only a single sealing element, for example as an integral component of the two sealing elements 94, 94′ shown, or also to provide more than two such sealing elements 94, 94′.

The housing 92 is formed from a housing element 96 and a closure element 98.

The housing element 96 has an essentially cup-shaped design, accommodates the at least one sealing element 94, 94′, and is nondetachably mounted on the base body 14. The at least one sealing element 94, 94′ extends, at least partially, in a chamber 100 which is enclosed and thus formed by the housing element 96 of the housing 92.

The closure element 98 has a cover- or cap-like design. The closure element 98 is provided with a central borehole 102 or similar opening having an approximately circular shape. A further instrument (not shown), in particular a gripping or other cutting instrument, preferably claw grasping forceps or a myoma drill, may be passed through the borehole 102, namely, via the borehole 102 through the proximal opening 90 in the base body 14, through the sealing device 88 at the proximal end 26 of the base body 14, through the base body 14 itself toward the distal end 16 of the base body 14, through the distal opening 18, and finally to the tissue (cylinder) to be removed in the surgical area. The closure element 98 in turn may be mounted on the housing element 96.

For this purpose the housing element 96 and the closure element 98 of the housing 92 may be joined together, either detachably or nondetachably, via a threaded connection or locking or snap connection 104. The constructive design of a detachable or nondetachable connection between the housing element 96 and the closure element 98 depends on the intended use and purpose of the apparatus 10 in the form of a single- or multiple-use apparatus, the design of the apparatus 10, and the selection of materials of which the individual components of the apparatus 10 according to the invention are composed, the intentions of the user, etc.

For a single use of the apparatus 10, the housing element 96 and the closure element 98 are nondetachably connected to one another via the threaded connection or locking or snap connection 104, by providing an adhesive, preferably a silicone adhesive, in between, or by ultrasonic welding. Thermal or chemical sterilization of the apparatus 10 and any multiple or further use of the apparatus 10 is thus prevented.

The housing 92 of the sealing device 88 and in particular the closure element 98 of the housing 92 is provided with a profile 105 on its circumference for manual actuation and rotation of the base body 14.

As previously mentioned, in the design in FIGS. 1 through 7B the sealing device 88 includes two sealing elements 94, 94′ which are adjacently situated in the housing 92 on the longitudinal axis 24 of the base body 14 of the cutting device 12, and which cooperate with one another.

One 94 of the two sealing elements 94, 94′ of the sealing device 88 essentially has the shape of a cone, conical frustum, cup, hat, or the like. The sealing element 94 includes a slotted opening 106 situated in a plane 108 (perpendicular to the plane of the page) which is offset with respect to a plane 112 (perpendicular to the plane of the page) defined by the base area 110 of the sealing element 36 [sic; 94] in the direction of the distal end 16 of the base body 14. The portion or section of the sealing element 94 essentially having the shape of a cone, conical frustum, cup, hat, or the like therefore extends in the direction of the distal end 16 of the base body 14, and projects into the chamber 100 of the housing 92. The sealing element 94 also has a radially outwardly extending peripheral section 114 which lies in the plane 112 defined by the base area 110 of the scaling element 94 and which is used for fixing in the housing 92.

The sealing element 94 has at least two sealing lips 116, 116′ which are in mutual operative engagement for closing and exposing the slotted opening 106. On account of the shape and positioning of the two sealing lips 116, 116′ of the sealing element 36 [sic; 94] in the housing 92 in the opposite direction of the action of a pressure artificially developed within a cavity of the body or a joint, for example by CO₂ installation, the sealing lips 116, 116′ are externally pressed with a complete seal against a further instrument (not shown), for example a gripping or other cutting instrument, in particular claw grasping forceps or a myoma drill. In this manner the sealing lips 116, 116′ are wedged, in a manner of speaking. The sealing lips 116, 116′ are pressed against the further instrument and remain in this position until the pressure is discontinued in a regulated manner. This ensures that the pressure necessary for operating, i.e., cutting or punching out, a tissue cylinder from a tissue and for subsequent removal of the cut or punched tissue cylinder from the surgical area is continuously maintained. Release of gas such as CO₂ or the like from the cavity of the body or a joint is effectively prevented.

The other 94′ of the two sealing elements 94, 94′ of the sealing device 88 has a disk-shaped design and is provided with a central, approximately circular opening 118. The previously mentioned further instrument (not shown), in particular a gripping or other cutting instrument, preferably claw grasping forceps or a myoma drill, may be passed through the opening 118 in the sealing element 94′. The central, approximately circular opening 118 is provided with an inner diameter which is less than or equal to an outer diameter of the further instrument.

As shown in FIG. 5B, the other sealing element 94′ is situated in the housing 92 on the longitudinal axis 24 of the base body 14, between the proximal end 26 of the base body 14 and the one sealing element 94. In particular, the other scaling element 94′ is (directly) adjacently associated with the plane 112 defined by the base area 110 of the one sealing element 94.

The two sealing elements 94, 94′ of the scaling device 88 may be fixed to one another in the housing 92 by means of a clamping disk 120 or the like.

According to FIGS. 1 and 8, the cutting elements 20, 20′ of the cutting devices 12, 12′ have a design which is essentially beveled or conical, or which extends obliquely outward facing away from the distal opening 18, 18′ and/or obliquely inward facing the distal opening 18. In the design of the apparatus 10 shown in FIG. 8, the cutting element 20 of the cutting device 12 has a design which is essentially beveled or conical, or which extends obliquely inward facing the distal opening 18. In contrast, the cutting element 20′ of the cutting device 12′ has a design which is essentially beveled or conical, or which extends obliquely outward facing away from the distal opening 18′. Although not specifically illustrated, any other given constructive designs or configurations of the cutting elements 20, 20′ relative to one another are also possible.

The cutting elements 20, 20′ may each have a circumferential or discontinuous smooth, serrated, or dentate face. In the design of the apparatus 10 shown in particular in FIG. 8, both cutting elements 20, 20′ are provided with a dentate face which has teeth distributed over the circumference with axially receding tooth bases and axially projecting tooth tips. Although not specifically illustrated, in this regard other given combinations may also be implemented. Thus, the cutting elements 20, 20′ of the two cutting devices 12, 12′ may have a serrated face, for example, having circular are sections distributed over the circumference with axially receding wave troughs and axially projecting wave crests.

The at least one, in particular the two, sealing element(s) 94, 94′ of the sealing device 88 is/are made of flexible plastic, in particular silicone.

The housing 32 of the gearing 30 and/or the centering and/or guide element and/or the output gear wheel 42 and/or one or both of the two input gear wheels 44, 46 and/or the support element 56 and/or the drive bushes 66, 66′ and/or the coupling elements 70, 70′ and/or the sealing device 88 and/or housing element 96 and/or the closure element 98 of the housing 92 and/or the clamping disk 120 is/are made of plastic, in particular polyoxymethylene, polyester, polyphenylene sulfone, ABS, acrylic, polycarbonate, tetrafluoroethylene or Impax, duroplastic elastomers with or without fiberglass reinforcement, or a combination thereof.

Instead, the housing 32 of the gearing 30 and/or the centering and/or guide element and/or the output gear wheel 42 and/or one or both of the two input gear wheels 44, 46 and/or the support element 56 and/or the drive bushes 66, 66′ and/or the coupling elements 70, 70′ and/or the sealing device 88 and/or housing element 96 and/or the closure element 98 of the housing 92 and/or the clamping disk 120 alternatively and/or additionally may be made of metal, in particular (rust-free) steel, stainless steel, aluminum, brass, zinc, red brass alloys, or an alloy of the above. On the other hand, the base bodies 14, 14′ of the cutting devices 12, 12′ and/or the coupling device 72 generally are preferably made of metal, in particular (rust-free) steel, stainless steel, aluminum, brass, zinc, red brass alloys, or an alloy of the above.

Although not specifically shown, the individual components of the gearing 30 situated between the at least two cutting devices 12, 12′ and the drive device 22 are designed and may be assembled in a modular fashion. The gearing 30 and therefore the entire apparatus 10 may thus be put together in a modular fashion by the operator or service personnel. On the one hand, the manufacturer may realize additional cost savings for installation. On the other hand, the apparatus according to the invention may thus be easily, quickly, and reliably disassembled, for example for disinfection or the like, and then reassembled for reuse. It is advantageous that the housing 32 of the gearing 30 or the housing element 34 and the housing cover 36 and/or the output gear wheel 42 and/or one or both of the two input gear wheels 44, 46 and/or the support element 56 are designed and may be assembled in a modular fashion.

The invention is not limited to the illustrated embodiments of the apparatus 10 corresponding to FIGS. 1 through 8. Accordingly, it is possible for the at least two cutting devices 12, 12′ for the illustrated exemplary embodiment of the apparatus 10 according to the invention to not be driven, or not driven exclusively, in counterrotation, but rather, alternatively or additionally to be driven in corotation if such a drive in the same direction were necessary for a surgical procedure. Although not specifically illustrated, in this case additional precautionary measures generally known as such would be provided with regard to the gearing 30, shifting possibilities for the gearing 30, etc. Furthermore, a locking or snap connection (not specifically illustrated) for mutual attachment of the housing element 96 and the closure element 98 may be provided instead of a threaded connection. The housing element 96 and the closure element 98 are joined together via at least one locking projection or similar catch lock on the housing element 96 or on the closure element 98 and at least one additional locking projection on the closure element 98 or on the housing element 96. The two locking projections or similar catch locks cooperate and may be brought into mutual engagement. In this regard, the at least one locking projection or similar catch lock and/or the at least one additional locking projection or similar catch lock may preferably be designed in such a way that the housing element 96 and the closure element 98 of the housing 92 are nondetachably connected to one another after engagement, or are not connectable to one another after subsequent disengagement. For example, one or both of the locking projections or similar catch locks may be provided with a predetermined breaking point in order to permanently impair the apparatus 10 after disengagement and thus make it impossible to be reused. The apparatus 10 according to the invention is equally well suited for single or multiple use.

1. Apparatus for cutting out and removing tissue cylinders from a tissue located within a cavity of the body or a joint and/or in or on a wall region thereof, comprising at least two, in particular two, cutting devices (12, 12′), each of which has a hollow cylindrical base body (14, 14′), a distal opening (18, 18′) at a distal end (16, 16′) of the base body (14, 14′), and a cutting element (20, 20′) which surrounds the distal opening (18, 18′), the at least two cutting devices being situated one inside the other and designed to be rotatable relative to one another, and comprising at least one drive device (22) for rotating the respective base body (14, 14′) of the at least two cutting devices (12, 12′) about the longitudinal axis (24, 24′) of the base body.

2. Apparatus according to claim 1, characterized in that the inner cutting device (12) is coaxially accommodated by the adjacent outer cutting device (12′) of the at least two cutting devices (12, 12′).

3. Apparatus according to claim 1 or 2, characterized in that the inner cutting device (12) is accommodated by the adjacent outer cutting device (12′) of the at least two cutting devices (12, 12′) with little play.

4. Apparatus according to one of claims 1 through 3, characterized in that the inner cutting device (12) is accommodated by the adjacent outer cutting device (12′) of the at least two cutting devices (12, 12′) with greater play, wherein at least one centering and/or guide element is situated between the base bodies (14, 14′) of the inner cutting device (12) and the adjacent outer cutting device (12′).

5. Apparatus according to one of claims 1 through 4, characterized in that the at least two cutting devices (12, 12′) may be driven by the drive device (22) in counterrotation and/or in corotation.

6. Apparatus according to one of claims 1 through 5, characterized in that gearing (30) which is accommodated in a housing (32) is provided between the at least two cutting devices (12, 12′) and the drive device (22).

7. Apparatus according to one of claims 1 through 6, characterized in that the gearing (30) includes an output gear wheel (42) associated with the drive device (22), and two input gear wheels (44, 46), in operative engagement with the one output gear wheel (42), which are each associated with one of the at least two cutting devices (12, 12′).

8. Apparatus according to one of claims 1 through 7, characterized in that the Output gear wheel (42) and the two input gear wheels (44, 46) of the gearing (30) are designed as bevel gears.

9. Apparatus according to one of claims 1 through 8, characterized in that the two input gear wheels (44, 46) which are each associated with one of the at least two cutting devices (12, 12′) are in simultaneous operative engagement with the output gear wheel (42) associated with the drive device (22), wherein the rotational axes (48, 48′) of the two input gear wheels (44, 46) coincide, and the rotational axes (48, 48′) of the two input gear wheels (44, 46) are each offset by ±90° relative to the rotational axis (50) of the output gear wheel (42).

10. Apparatus according to one of claims 1 through 9, characterized in that the output gear wheel (42) is detachably mounted on a support element (56) which is rotatably supported on the housing (32) of the gearing (30).

11. Apparatus according to one of claims 1 through 10, characterized in that the output gear wheel (44) and the support element (56) are detachably joined together by a locking or snap connection or a threaded connection.

12. Apparatus according to one of claims 1 through 11, characterized in that the output gear wheel (42), in particular the support element (56), together with the drive device (22) is connected in a rotationally fixed manner via a peg-shaped transmission element (64) to a profile having a rectangular, square, hexagonal, or otherwise polygonal, elliptical, trapezoidal, parallelogram-like, or semicircular shape.

13. Apparatus according to one of claims 1 through 12, characterized in that the two input gear wheels (44, 46) are each accommodated by drive bushes (66, 66′) which are respectively associated with one of the at least two cutting devices (12, 12′).

14. Apparatus according to one of claims 1 through 13, characterized in that the two input gear wheels (44, 46) are each attached to the drive bushes (66, 66′) via a threaded connection or a locking or snap connection.

15. Apparatus according to one of claims 1 through 14, characterized in that the drive hushes (66, 66′) project through the housing (32) of the gearing (30).

16. Apparatus according to one of claims 1 through 15, characterized in that the drive device (22) may be controlled and/or regulated using a control and/or regulation device which in particular is electronic, in such a way that the at least two cutting devices (12, 12′) may each be driven by the drive device (22) at intervals in the opposite drive direction.

17. Apparatus according to one of claims 1 through 16, characterized in that the time intervals for driving the at least two cutting devices (12, 12′) in the opposite drive direction may be set and/or preset by a control and/or regulation device which in particular is electronic.

18. Apparatus according to one of claims 1 through 17, characterized in that the at least two cutting devices (12, 12′) are each provided with coupling elements (70, 70′) at the proximal end (26, 26′) and/or at the region (28, 28′) of the base body (14, 14′) facing the proximal end (26, 26′), the coupling elements being nondetachably connected to the respective base body (14, 14′).

19. Apparatus according to one of claims 1 through 18, characterized in that the at least two cutting devices (12, 12′) and the drive device (22) together with the gearing (30) may each be connected in a rotationally fixed manner via a coupling device (72).

20. Apparatus according to one of claims 1 through 19, characterized in that the coupling device (72) is designed as a plug-in, locking, or snap device.

21. Apparatus according to one of claims 1 through 20, characterized in that the coupling device (72) has a rocker arm (74) which is mounted in each case on the drive bushes (66, 66′) and the housing (32) of the gearing (30) so as to be pivotable about a rotational axis (76), at one end (78) is acted on by a spring (80), and at an end (82) opposite from the one end includes an approximately radially situated pin (84), bolt, or projection for engaging in a correspondingly designed borehole (86) or recess in the coupling element (70, 70′) of the at least two cutting devices (12, 12′) or the drive device (22).

22. Apparatus according to one of claims 1 through 21, characterized in that the coupling element (70) of the inner/innermost of the at least two cutting devices (12, 12′) is designed as a sealing device (88) for closing and exposing a proximal opening at the proximal end (26) of the base body (14).

23. Apparatus according to one of claims 1 through 22, characterized in that the sealing device (88) has a two-part, essentially cup-shaped housing (92) with a housing element (96) mounted on the base body (14), and a lid- or cap-shaped closure element (98) which may be fastened to the housing element (96).

24. Apparatus according to one of claims 1 through 23, characterized in that the housing element (96) and the closure element (98) of the housing (92) are detachably or nondetachably connected to one another via a threaded connection or a locking or snap connection (104).

25. Apparatus according to one of claims 1 through 24, characterized in that the housing element (96) and the closure element (98) of the housing (92) are nondetachably joined together by providing an adhesive, preferably a silicone adhesive, in between, or by ultrasonic welding.

26. Apparatus according to one of claims 1 through 25, characterized in that the housing element (96) and the closure element (98) of the housing (92) are connected to one another via at least one locking projection or similar catch lock on the housing element (96) or on the closure element (98), and via at least one additional locking projection or similar catch lock and at least one catch recess or the like on the closure element (98) and the housing element (96), respectively, which cooperate and may be brought into mutual engagement.

27. Apparatus according to one of claims 1 through 26, characterized in that the at least one locking projection or similar catch lock is provided on the housing element (96) or on the closure element (98), and the at least one additional locking projection or similar catch lock and the at least one catch recess or the like are provided on the closure element (98) and the housing element (96), respectively, in such a way that the housing element (96) and the closure element (98) of the housing (92) are nondetachably connected to one another after engaging the at least one locking projection or similar catch lock and the at least one additional locking projection or similar catch lock, or the at least one catch recess, or are not connectable to one another after disengaging the at least one locking projection or similar catch lock and the at least one additional locking projection or similar catch lock, or the at least one catch recess.

28. Apparatus according to one of claims 1 through 27, characterized in that the sealing device (88) includes two sealing elements (94, 94′) which are adjacently situated in the housing (92) on the longitudinal axis (24) of the base body (14) of the cutting device (12), and which cooperate with one another.

29. Apparatus according to one of claims 1 through 28, characterized in that one (94) of the two sealing elements (94, 94′) of the sealing device (88) essentially has the shape of a cone, conical frustum, cup, hat, or the like, and includes a slotted opening (106) situated in a plane (108) which is offset with respect to a plane (112) defined by the base area (110) of the sealing element (94) in the direction of the distal end (16) of the base body (14) of the cutting device (12), and for fixing in the housing (92) has a radially outwardly extending peripheral section (114) in the plane (112) defined by the base area (110) of the sealing element (94).

30. Apparatus according to one of claims 1 through 29, characterized in that one (94) of the two sealing elements (94, 94′) has at least two sealing lips (116, 116′) which are in mutual operative engagement for closing and exposing the slotted opening (106).

31. Apparatus according to one of claims 1 through 30, characterized in that the other (94′) of the two sealing elements (94, 94′) of the sealing device (88) has a disk-shaped design, and has a central, approximately circular opening (118) through which a further instrument, in particular a gripping or other cutting instrument, preferably claw grasping forceps or a myoma drill, may be passed.

32. Apparatus according to one of claims 1 through 31, characterized in that the central, approximately circular opening (118) in the other (94′) of the two sealing elements (94, 94′) has an inner diameter which is less than or equal to an outer diameter of the further instrument.

33. Apparatus according to one of claims 1 through 32, characterized in that the other (94′) of the two sealing elements (94, 94′) in the housing (92) is situated on the longitudinal axis (24) of the base body (14) of the cutting device (12), between the proximal end (26) of the base body (14) and the one (94) of the two scaling elements (94, 94′), in particular adjacent to the plane (112) defined by the base area (110) of the one (94) of the two sealing elements (94, 94′).

34. Apparatus according to one of claims 1 through 33, characterized in that the at least one, in particular the two, sealing element(s) (94, 94′)′ of the sealing device (88) is/are made of flexible plastic, in particular silicone.

35. Apparatus according to one of claims 1 through 34, characterized in that the cutting elements (20, 20′) of the at least two cutting devices (12, 12′) essentially have a design which is beveled, conical, or tapered, or which extends obliquely outward facing away from the distal opening (18, 18′) and/or obliquely inward facing the distal opening (18, 18′).

36. Apparatus according to one of claims 1 through 35, characterized in that the cutting elements (20, 20′) of the at least two cutting devices (12, 12′) have a circumferential or discontinuous smooth, serrated, or dentate lace.

37. Apparatus according to one of claims 1 through 36, characterized in that the cutting elements (20, 20′) of the at least two cutting devices (12, 12′) with the serrated face have circular are sections distributed over the circumference with axially receding wave troughs and axially projecting wave crests.

38. Apparatus according to one of claims 1 through 37, characterized in that the cutting elements (20, 20′) of the at least two cutting devices (12, 12′) with the dentate face have teeth distributed over the circumference with axially receding tooth bases and axially projecting tooth tips.

39. Apparatus according to one of claims 1 through 38, characterized in that the drive device (22) is designed as a motor which may be driven electrically, by battery, pneumatically, or hydraulically.

40. Apparatus according to one of claims 1 through 39, characterized in that the housing (32) of the gearing (30) and/or the centering and/or guide element and/or the output gear wheel (42) and/or one or both of the two input gear wheels (44, 46) and/or the support element (56) and/or the drive bushes (66, 66′) and/or the coupling elements (70, 70′) and/or the sealing device (88) and/or housing element (96) and/or the closure element (98) of the housing (92) and/or the clamping disk (120) is/are made of plastic, in particular polyoxymethylene, polyester, polyphenylene sulfonic, ABS, acrylic, polycarbonate, tetrafluoroethylene or Impax, duroplastic elastomers with or without fiberglass reinforcement, or a combination thereof.

41. Apparatus according to one of claims 1 through 40, characterized in that the base bodies (14, 14′) of the at least two cutting devices (12, 12′) and/or the housing (32) of the gearing (30) and/or the centering and/or guide element and/or the output gear wheel (42) and/or one or both of the two input gear wheels (44, 46) and/or the support element (56) and/or the drive bushes (66, 66′) and/or the coupling elements (70, 70′) and/or the coupling device (72) and/or the sealing device (88) and/or housing element (96) and/or the closure element (98) of the housing (92) and/or the clamping disk (120) is/are made of metal, in particular (rust-free) steel, stainless steel, aluminum, brass, zinc, red brass alloys, or an alloy of the above.

42. Apparatus according to one of claims 1 through 41, characterized in that the individual components of the gearing (30) situated between the at least two cutting devices (12, 12′) and the drive device (22) are designed and may be assembled in a modular fashion.

43. Apparatus according to one of claims 1 through 42, characterized in that the housing (32) of the gearing (30) or the housing element (34) and the housing cover (36) and/or the output gear wheel (42) and/or one or both of the two input gear wheels (44, 46) and/or the support element (56) and/or the drive bushes (66, 66′) are designed and may be assembled in a modular fashion.

44. Use of an apparatus according to one of the preceding claims for laparascopic, thoracoscopic, or arthroscopic minimally invasive surgical procedures.
